(19) United States
(12) Patent Application Publication
(10) Pub. No.: US 2016/0369611 A1
(43) Pub. Date: Dec. 22, 2016

(54) HYDROCARBON FRACTURING PROCESS

(71) Applicant: U.S. Flare Management, Palm Springs, CA (US)

(72) Inventor: Mark Allen Bragg, Palm Springs, CA (US)

(21) Appl. No.: 15/184,734

(22) Filed: Jun. 16, 2016

Related U.S. Application Data
(60) Provisional application No. 62/180,536, filed on Jun. 16, 2015.

(51) Int. Cl. E21B 43/40; C10L 1/00; C07C 9/08; F25J 1/00; C07C 9/00; C10L 3/06; C09K 8/62; E21B 43/26; C07C 9/10

(52) U.S. Cl. CPC E21B 43/40; E21B 43/26; C10L 1/00; C07C 9/08; C07C 9/10; C07C 9/00; C10L 3/06; C09K 8/62; F25J 1/0022; C10L 2200/0423; F25J 2260/60

(57) ABSTRACT

The present invention relates to method for collecting hydrocarbons from a well site for later use. Specifically, the invention aims to use a mixture containing at least some liquefied natural gas (LNG) at the wellsite. The LNG could be used as a component of hydraulic fracturing fluid or power an on-site generator, among other uses.

### BACKGROUND OF THE INVENTION

This invention claims priority under 315 U.S.C. §119(e) to U.S. Provisional Application No. 62/180,536, filed on Jun. 16, 2015.

### FIELD OF THE INVENTION

This invention relates to fracturing processes and fluid formulations for obtaining hydrocarbons.

Petroleum is typically produced by drilling into underground rock formations where oil is contained in the gaps between the grains of porous rock. Often the pressure of overlying rock is sufficient to push oil up through the production well. Various means of have been developed to enhance the flow of oil to the well such as by pumping, or by injecting water, steam, detergents and other flow-enhancing chemicals. In other situations, mixtures of fluids are injected to create additional cracks and/or widen pores in the rock to enhance flow in a process known as hydraulic fracturing. This process typically uses fluid formulations comprised largely of water with addition of other chemicals to enhance and maintain the widening of gaps in the porous rock. Associated petroleum gas (APG), a form of natural gas, is a term used to describe light hydrocarbons produced as a byproduct of petroleum production APG is found with deposits of petroleum, often either dissolved in the oil or as a free gas cap above oil in a reservoir, or intermingled with hydrocarbon-containing sand or rock formations. APG may be recovered from the petroleum site, released as a waste product or simply burnt off into the atmosphere, The burning process is also known as flaring, and the APG gas that is burnt off is known as flare gas.

Flare gas typically contains light hydrocarbons (typically in the range of 1 to 7 carbon atoms per molecule), and may also contain other gases such as carbon dioxide, nitrogen, hydrogen sulfide, particulate matter and ash. Flare gas is often considered a waste product where there is no local market for the gas or no pipeline or other means to transport the gas to a useable location or market.

One way to avoid burning of flare gas is to recover and convert it to liquefied natural gas (LNG). LNG is typically produced by refrigerating the gas to very low temperatures where the components become liquid at normal atmospheric pressures. LNG may be made in a liquefaction plant at or near the oil reserve and transported to an area where fossil fuel resources are needed or have higher value than near the production site. For example LNG is exported from locations such as Algeria, Egypt, Nigeria, Angola, Oman, Qatar, Yemen, Russia, Trinidad and Tobago, Australia, Malaysia, and Indonesia where the local markets are unable to consume the natural gas produced. In these locations the price of natural gas is low because there is an abundant supply and low local demand. The low price of natural gas and the enhanced value of the higher-demand markets offsets the expense of building an LNG liquefaction plant and transporting the LNG.

Markets where LNG may be in demand include Japan, South Korea and Taiwan. These areas have very high populations and very little access to domestic fossil fuel resources. LNG provides these populations with a clean-burning fuel that is economical and easy to distribute through pipelines.

LNG is typically received on the market-side at dedicated terminal facilities designed to offload the LNG and convert it back to gaseous form in a process known as regasification. Once regasified the gas may be sent by pipeline for distribution or placed in temporary storage as needed.

The advent of utilization of very low porosity petroleum reservoirs such as shale fields has led to an increase in the production of APG and consequent increases in flaring. Because hydraulic fracturing is typically used to enhance the flow of petroleum from these resources, a substantial increase in APG production and flaring has occurred in areas where hydraulic fracturing is being done. Wasteful burning of natural gas derived from hydraulic fracturing processes is a growing problem. Oil and gas production from shale fields across the nation, including those located in the Bakken, Marcellus, Haynesville, Fayetteville, and Barnett regions have increased significantly. The problem of wasting flare gas also has grown.

For example, the Bakken Basin, located in North Dakota, contains a large shale oil formation in which gas is naturally trapped within the shale rock. The rapid rise in oil drilling and production in this area has led to an increase in the production of unused natural gas. When the gas cannot be recovered or processed it is burnt off in the atmosphere. Significant amounts of gas produced by a hydraulic fracturing process have been burnt off, resulting in a significant waste of gas and energy and loss of revenue to governments, taxpayers, resource and land owners and producers.

Flaring can also lead to increased pollution by creating greenhouse gases or by emitting products of incomplete combustion such as soot, black carbon, and incompletely combusted gas components of the APG. State and national regulatory agencies have responded by tightening environmental controls and developing regulations to reduce flaring.

The increased production of unused gas also has negatively affected the production and use of LNG. High production volumes of natural gas have caused natural gas prices to fall significantly. As a result, terminals that were previously built to receive liquefied natural gas are now underutilized or idle. This can lead to shortages in availability of this form of natural gas.

Accordingly, there is a need to better utilize APG It would benefit the environment to curtail flaring and to develop new uses for gas associated with fracturing and other process in which flare gas is burnt off. It also would be economically beneficial to the industry to convert unused gas associated with fracturing to usable gas and gas products such as LNG. In addition, there is a need to enhance petroleum production and reduce wasted gas by converting the gas to LNG and using it to inject into fracturing operations at the wellhead.

These and other needs may be met by converting flare gas on site and reusing or storing the converted gas for later use. For example, these needs may be met by converting potentially wasted flare gas to LNG at or near the gas production site and using the converted LNG with an ongoing operation, such as a fracturing operation, as a power source or a fracturing fluid.

### BRIEF SUMMARY OF THE INVENTION

It has now been discovered that recovery and use of gas produced from oil and gas producing processes, such as a fracturing process, can provide benefits to the hydrocarbon industry. It has also been discovered that recovered gas may be used in its natural or substantially natural form or it may be converted to other forms and then used. It has also been discovered that regardless of form, recovered gas may be used immediately or stored for future use, or it may be modified, stripped or otherwise altered for present use or storage.

In one embodiment, for example, on-site recovery and reuse of gas from or near a fracturing well may be converted to LNG providing significant economic and environmental advantages to a user. Production of an LNG product from gas recovered from a fracturing site may be recycled for use as a fracturing fluid at the production site or a nearby fracturing site. Alternatively, a portion of the gas recovered from a fracturing site may be used to power machinery while another portion may be converted into LNG for other uses, transportation or storage for subsequent use on site or at nearby fracturing sites.

The invention and its use in exemplary embodiments will be described in greater detail in the following drawings and passages. These drawings and descriptions illustrate different aspects and applications of the invention and are not intended to limit the full scope and spirit of the invention.

### BRIEF DESCRIPTION OF THE DRAWINGS

FIG. 1 is a process flow diagram of an oil well operation and exemplary use of associated gas.

### DETAILED DESCRIPTION OF ILLUSTRATIVE EMBODIMENTS

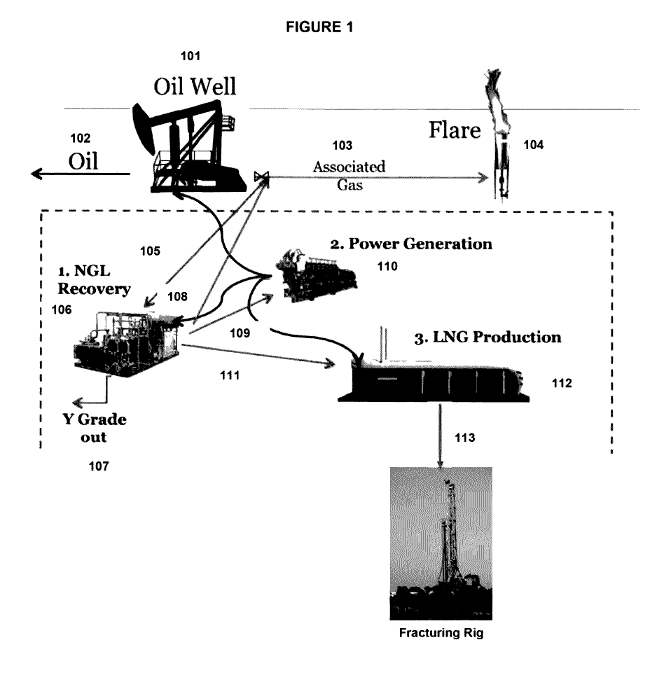

FIG. 1 illustrates an oil production process in which well 101 produces oil that is transported from the well in line 102 and associated gas that is transported from the well in line 103. Associated gas may be flared at flare pipe 104. All or portions of the gas in line 103 may be fed away in one or more lines 105 for recovery operations and later use, including those described in this document.

Oil production processes associated with this invention may be any known process for extracting oil from the earth. Such processes include pumping oil from a reservoir and extracting oil through a fracturing operation. Wells that produce oil and gas may be drilled, vertically, horizontally and in combinations thereof. These wells may receive fracturing treatment, including hydraulic fracturing or fracturing with fluids that include hydrocarbons.

While the associated gas useful in this invention may typically originate from an oil reservoir or formation that is drilled, the gas may be derived from any source including conventional and unconventional oil or gas reservoirs. Such sources include porous formations such as sandstone as well as ‘light’ formations such as carbonate or shale reservoirs for oil and gas and also may include natural gas found in other hydrocarbon-bearing formations such as coal beds. The gas contemplated in this invention also may be sourced in pipelines containing gas that originated from any conventional or unconventional oil or gas reservoir.

Thus, the gas may be sourced from drilling operations in conventional or unconventional oil reservoirs. These reserves include shale gas, tight gas, tight oil, and coal seam gas wells. Gas thus may be sourced from sands and carbonates such as limestone or dolomite where the oil or gas is contained in interconnected pore spaces. The gas also may be found in organic rich shale formations located near porous and permeable sandstone or carbonate, or it may appear in tight sands and carbonates coal, and shale where gas ay be sourced from spaces within or between the formations or from the reservoir rock itself. Due to the low permeability of such formations, additional permeability may be created by stimulating the reservoir through known means such as fracturing, including hydraulic fracturing.

In the case of shale, gas may be found in a local macro-porosity system within the shale, or within the micro pores of the shale, or it may be adsorbed onto minerals or organic matter within the shale.

Shale oil and gas wells may be similar to other conventional and unconventional wells in terms of depth, production rate, and drilling. Active shale deposits, for example, include the Bakken Shale, Barnett Shale, the Antrim Shale, the Marcellus Shale, and the New Albany Shale. Each of these shale basins is different and each has a unique set of exploration criteria and operational challenges. Because of these differences, the development of shale gas resources in each of these areas faces unique challenges.

This invention is particularly adaptable for gasses sourced from fracturing treatments to oil and gas reservoirs such as that found in the Bakken formation. For example, hydraulic fracturing may involve pumping a fracturing fluid under high pressure into a shale formation to generate fractures or cracks in the target rock. This process allows the natural gas contained within the formation to flow to the well in economic quantities.

Fracturing treatments include the, use of fluids, which may be primarily water mixed with a wide range of additives that enhance the movement of fluids, the creation of widened pores and cracks and the maintenance of the widened spaces (using additives known as proppants). While water and sand make up a significant quantity of fracture fluids used today, this invention is useful with other fluids which may comprise hydrocarbons and other proppants which may comprise metal oxides, ceramics, polymers and other chemicals and combinations of any of these. Various chemical additives may be added to the fracturing fluid. These additives provide improvement of the efficiency of the fracture job and include acids, salts, polymers, acrylamides, friction reducers, glycols, carbonates, glutaraldehyde, guar gum, and alcohols. These additives may be used in one or more combinations to clean, disinfect, create or sustain gels, lubricate, prevent scale or corrosion, adjust viscosity, or winterize the process.

As already noted, all or portions of the gas in line 103 may be fed away in one or more lines 105 for recovery operations and later use. According to the invention, the gas may stripped to remove valuable liquid hydrocarbon and/or utilized many different ways. For example, the gas may be converted into liquid natural gas (LNG) for immediate use or storage and later use as is or in natural gas distribution networks, it may be used for on-site electricity generation with engines or turbines, it may be re-injected into a fracturing treatment for enhanced oil or gas recovery, or it may be used as feedstock for petrochemical processes.

As FIG. 1 illustrates, in an example embodiment associated gas from the oil well is charged to natural gas liquid (NGL) recovery unit 106 via line 105. The NGL recovery unit may be any unit operation suitable for separating liquid hydrocarbons from gas. Suitable units include but are not limited to those manufactured by BFXCO, GTuit, Pioneer Resources, and Kinder-Morgan.

Preferably NGL recovery unit 106 is portable and appropriately sized to match the volumes and compositions of gas produced at the particular production site. Appropriate sizing allows the NGL unit to be located at or near the oil and gas wellbore site and to be moved to other sites as gas availability decreases at the initial location. A portable and appropriately located NGL recovery unit provides the ability to avoid a significant portion of wasteful flaring and provides a high value product for either local use or export to nearby, regional, national or international markets. Example products of light gasoline, and propane, butane or mixtures of these such as ‘Y-grade’. These products may also be used locally to provide fuel or to generate power that may be used at the well site.

In this embodiment, NGL recovery unit 106 produces Y-grade hydrocarbons in effluent line 107 while hydrocarbon fuel is fed through line 106 to power generation unit 110. Effluent from NGL recovery unit 106 also feeds liquid natural gas (LNG) production unit 112. The NGL recovery unit 106 may also produce light gas that may be recycled to gas line 103 for disposal or flaring.

Power generation unit 110 is typically one that consumes hydrocarbons to produce electricity. Power generation unit 110 preferably generates power from hydrocarbon effluent emitted from NGL recovery unit 106 via line 109. Power generation unit 110 may supply power to one or more units of power consuming equipment in oil well 101, NGL recovery unit 106 and LNG production unit 112. The power generation unit 110 may be any unit operation suitable for converting and generating power from the combustion of hydrocarbons.

Suitable power generation units include but are not limited to GE, Capstone, and CAT power generation units.

LNG production unit 112 produces liquefied natural gas for use, storage or transport for later use.

LNG production unit 112 may be any unit operation suitable for converting hydrocarbon gas, particularly natural gas, to liquefied gas. Suitable units include but are not limited to Galileo Cryobox, Dresser-Rand LNGO and others currently under development.

The hydrocarbon content of the associated gas in lines 103 and 105. Y-grade in line 105, and NGL effluent in lines 109 and 111 may vary depending on many factors, including the oil and gas components exiting the well bore, the operating parameters of the NGL recovery unit 106, power generation unit 110 and LNG production unit 112. Typical hydrocarbons present in associated gas line 103 and 105 range from C1 to C7 and preferably from C1 to C5, including straight and branched chain and isomers. Typical hydrocarbons present in Y-grade line 105 range from C2 to C5 and preferably C3 to C4. Typical hydrocarbons present in NGL effluent in lines 109 and 111 range from C2 to C7 and preferably C4 to C6.

It is a feature of the invention that one or more of the NGL recovery, power generation and LNG production units is located at or near the site of the oil well and flare gas operations. It is a further feature of the invention that one or more of the NGL recovery, power generation and LNG production units is portable or capable of being assembled and disassembled at one location and reassembled at a different location.

These mobility features make it possible to efficiently and economically reduce or eliminate the burning of flare gas and converting gas to use in oil producing operations. The mobility and compatibility of these unit operations make it possible for oil production wells located in remote areas to reduce or eliminate flaring of APG, increase the creation of valuable products, and reduce environmental impacts.

Such remote operations typically have no economic access to pipelines to transport associated field gas for processing or other use. Such remote operations will experience significant economic gain by deploying gas recovery and processing operations that are mobile, portable and located at or near the site of the oil well.

These unit operations may be used in any combination and under many different conditions. For example, in another embodiment of the invention, associated gas first is stripped of hydrocarbons which are retained for other use. The resulting gas is converted to liquid gas at or near the location of the gas source or oil well reservoir. The liquid gas is then used to provide power to drilling or other processing equipment either onsite on in other operations in the surrounding area. Alternatively, the liquid gas may be stored and used as a fluid for fracturing treatments occurring at or near the location of the liquid gas conversion operation.

Another embodiment of the invention is a process in which liquid hydrocarbon is used as a fluid for hydrocarbon fracturing operation. In this embodiment, a rig produces oil and associated gas. All or a portion of associated gas is normally fed to a flaring operation and diverted to an NGL production unit. Hydrocarbon effluent is removed from the NGL production unit and is fed to a power generation unit. Light or stripped gas from the power generation unit may be recycled to an associated gas line for further processing or flaring if necessary.

Hydrocarbon gas effluent from the NGL unit is fed to the LNG unit. As previously described, the LNG unit converts gaseous hydrocarbons, preferably primarily methane gas, to liquefied gas hydrocarbons, preferably LNG. Effluent from the LNG unit may be fed to a storage unit for later use. Alternatively, effluent LNG is also directly fed to a fracturing rig, as shown in FIG. 1; 113. LNG from the production unit may also be combined with LNG from the storage tank.

As noted, the liquefied hydrocarbon may be LNG or liquefied hydrocarbons of various compositions. The exact composition of liquefied hydrocarbon varies depending on the components of oil and gas extracted from the oil well, the desired amounts and hydrocarbon compositions in the feed and effluents for the NGL, power generation and liquefied hydrocarbon gas production units.

The liquefied hydrocarbons present in the fracturing fluid may comprise any number of carbon-containing components that are suitable to be liquefied. Preferably, the contemplated fracturing fluid includes from one to four carbons, including isomers, and most preferably CH₄ , C₂H₆, and mixtures thereof. The liquefied hydrocarbons are typically maintained at atmospheric pressure and at a temperature of −240F to −260F, preferably −260F.

The liquefied hydrocarbon may be used in one or more staging and sub-staging operations of a fracturing process.

Use of liquefied hydrocarbons as a fracturing fluid saves water and space required for hydraulic fracturing. Drilling and hydraulic fracturing typically requires very large volumes of water often of the order of 2 to 4 million gallons per well. Substituting all or part of this water with otherwise wasted natural gas can represent considerable savings of a valuable resource, and reductions in the cost of water storage, handling and use facilities at the fracturing site as well as reducing the cost and risks associated with disposal.

1. A method of extracting hydrocarbon from the ground comprising:
recovering gas from a hydrocarbon producing wellsite;
converting recovered gas to a liquefied gas; and
feeding at least a portion of the liquefied gas to a fracturing operation nearby the wellsite.

2. The method of claim 1, wherein at least a portion of the liquefied gas is exported to another fracturing operation.

3. A formulation for fracturing fluid comprising liquefied gas wherein at least a portion of the liquefied gas comprises methane extracted from the fracturing operation site.

4. A method of extracting hydrocarbon from the ground consisting of:
recovering a hydrocarbon from a hydrocarbon producing wellsite.

5. The method of claim 4 wherein the hydrocarbon is selected from a group consisting of light gasoline, propane, and butane.

6. The method of claim 4 wherein the hydrocarbon is Y-grade hydrocarbons.

7. The method of claim 4 wherein the hydrocarbon is used to power on-site generators.

8. The method of claim 4 wherein the hydrocarbons are stored for later use at the site.

9. The method of claim 4 wherein the hydorcarbons are exported to other fracturing sites.
